(19) United States
(12) Patent Application Publication
(10) Pub. No.: US 2011/0192986 A1
(43) Pub. Date: Aug. 11, 2011

(54) ELECTRON BEAM STERILIZING DEVICE

(76) Inventors: Kurt Holm, Baden (CH); Werner Haag, Lugnorre (CH); Dominique Cloetta, Villars-sur-Glane (CH)

(21) Appl. No.: 13/122,902

(22) PCT Filed: Sep. 25, 2009
(86) PCT No.: PCT/EP09/06941

§ 371 Date: Apr. 6, 2011

Related U.S. Application Data
(60) Provisional application No. 61/173,484, filed on Apr. 28, 2009.

(30) Foreign Application Priority Data
Oct. 7, 2008 (SE) 0802101-6

(51) Int. Cl. H01J 3/14; A61L 2/02

(52) U.S. Cl. 250/396 R

(57) ABSTRACT

An electron beam sterilizing device comprises an electron-generating filament, a grid connected to a voltage source, a beam shaper, and an output window. A high voltage source generates a high voltage potential between the electron-generating filament and the output window, for acceleration of electrons. The usability of the device is enhanced in that the electron-generating filament and/or the grid electrode comprises at least two operational portions for variation of the current and form of an output electron beam.

### TECHNICAL FIELD

The present invention relates to an electron beam sterilizing device and in particular to such device adapted for sterilization of containers.

### TECHNICAL BACKGROUND

Electron beam sterilizing devices are known, which are lowered into a package to be sterilized. Emission of electrons onto the walls of the package sterilizes it. The level of sterilization is determined by the irradiation dose delivered onto the wall. If the delivered dose is too small the sterilization will not be adequate, and if the dose is too high the package material might be affected negatively. The negative effects include that the taste of the final product in the package might be affected (off-taste problem) and that the package material might be deformed and/or damaged. The off-taste problem is obviously a problem to consider if the package is to be used as a container for foodstuff, such as beverage.

The irradiation dose will be affected by, among other things, the irradiation intensity and the irradiation time. It will also be affected by the distance between the electron beam sterilizing device exit and the package wall to be irradiated.

In a situation where all parameters can be varied without constraint, the problem of sterilizing packages by means of an electron beam is not a difficult task. However, in a modern foodstuff processing plant, where thousands and thousands of packages are to be manufactured, sterilized, filled, and sealed in a rapid pace, the conditions are quite different. For instance, the required pace is high, and the sterilising machine thus has to operate fast. Also, the shapes of the packages may not be uniform, in that the typical package comprises a neck portion where the cap is located, a tapering shoulder portion, and a body portion, terminated by the bottom of the container, meaning that the cross section of the package will vary over its length. The cross sectional shape may be circular, quadratic or rectangular, with or without rounded corners, racetrack shaped etc. This will in turn result in difficulties in obtaining adequate and equal irradiation on all surfaces of the package.

### SUMMARY OF THE INVENTION

It is an object of the present invention to eliminate or alleviate the above problems by providing an improved electron beam sterilizing device in accordance with the independent claims. Preferred embodiments are defined by the dependent claims. In the following the term “beam shape” relates to the beam-intensity profile (beam profile) in a direction perpendicular to the direction of propagation.

### BRIEF DESCRIPTION OF THE DRAWINGS

FIG. 1 is a cross sectional view of an electron beam sterilizing device arranged in a package and irradiating the same.

FIG. 2 is a schematic view of a sterilization device by the present applicant.

FIG. 3 is a schematic cross section of a sterilization device in accordance with a first embodiment of the present invention.

FIGS. 4-6 are partial views of FIG. 3, showing different modes of operation.

FIGS. 7-9 are partial views, similar to those of FIG. 4-6, showing different modes of operation for a sterilization device according to a second embodiment of the present invention.

FIG. 10 is a partial view, similar to FIGS. 7-9 of a sterilization device according to a third embodiment of the present invention.

### DESCRIPTION OF EMBODIMENTS

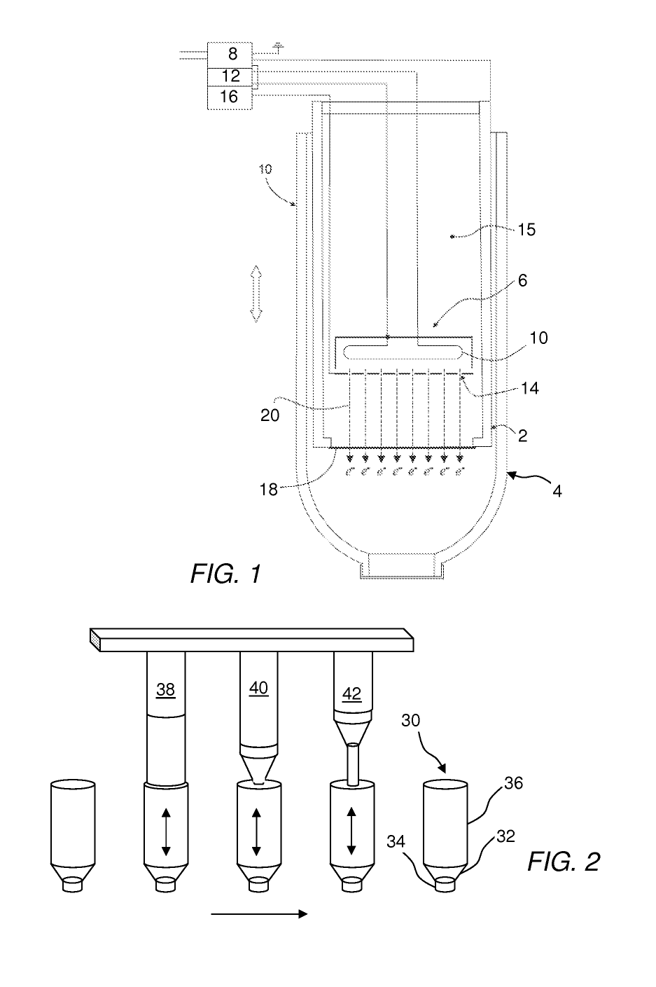

A brief description of electron beam sterilization will be given in the following, referring to FIG. 1. FIG. 1 illustrates an electron beam sterilizing device 2, or emitter, arranged in a package 4. It operates basically as an electron gun and generally comprises an electron beam generator 6, which is coupled to a high voltage supply 8. The generator 6 has a filament 10, which is forming the free electrons, and the filament is connected to a filament power supply 12 for this purpose. The filament is generally made of tungsten, and its basic function is that when it is heated to an elevated temperature, such as in the order of 2000° C., a cloud of electrons e⁻ are emitted.

There is a grid 14 adjacent to the filament 10 and by applying or not applying a positive or negative voltage to the grid 14 by means of the grid control supply 16 the electrons formed at the filament 10 will exit the grid 14, or not. Said components are located in a vacuum chamber 15.

At the output end of the device 2 an exit window 18 is arranged, and on their way to the exit window the electrons are accelerated in a high-voltage field. The potential difference in the high-voltage field is generally below 300 kV and for the inventive purposes it will be in the order of 70-120 kV, resulting in a kinetic energy of 70-120 keV for each electron in the electron beam 20, before passing the exit window 18. The exit window 18 is generally a metallic foil, such as titanium, having a thickness of 4-12 μm, which is supported by supporting net (not shown) made of aluminium or copper or any other suitable material. The supporting net prevents the foil from collapsing as a result of the vacuum inside the device. Further, the supporting net acts as a heat sink or a cooling element, such that it transports heat away from the foil, generally by conducting it to a cooling fluid, such as a cooling fluid line. Aluminium has a tendency to degrade during the conditions present in a production process, which is why copper is the preferred alternative for the purposes of the described application, but other alternatives are possible.

Once leaving through the exit window 18 the electrons 20 will have an optimal working distance (in this case working radius) of 5-50 mm, in air at normal pressure and temperature, following a Brownian motion, for the mentioned energy range. Some specific examples include 5 mm for a voltage of 76 kV, and 17 mm for a voltage of 80-82 kV, with a sterilisation depth of about 10 μm. This implies that when sterilizing a package, the emitter has to be lowered into the package to achieve a proper irradiation. By altering the atmosphere in the surrounding environment around the emitter the working distance may be altered. Reducing the pressure with 50% will basically double the working distance, and exchanging the gas in the atmosphere from air to nitrogen or helium will also affect the working distance, in a predictable fashion.

In the previous and following description similar components share the same last two digits in the reference numbers, and if the properties are similar, these will not be repeated.

FIG. 2 illustrates a solution disclosed in a patent application by the present applicant. The fundamental problem is similar to what is the case for the present application, the problem of sterilizing packages having a non-uniform cross section. In the device of FIG. 2 the packages 30 to be sterilized are so-called ready to fill (RTF) packages. RTF-packages are generally sterilized after the shoulder portion 32, comprising the cap 34 (or opening device) has been attached to the body portion 36. Following the sterilization the packages 30 are filled, through the bottom (facing upwards), after which the bottom is sealed off. As apparent from FIG. 2 that particular sterilization device comprises three emitters 38, 40, 42. Each emitter is adapted to irradiate a particular portion of the package 30. The one 38 to the left irradiates the body portion 36, the one 40 in the middle the shoulder portion 32 and the one 42 to the right the opening device 34. In this way the package 30 will be exposed to a sufficient irradiation dose, while no surface will be exposed to too much irradiation.

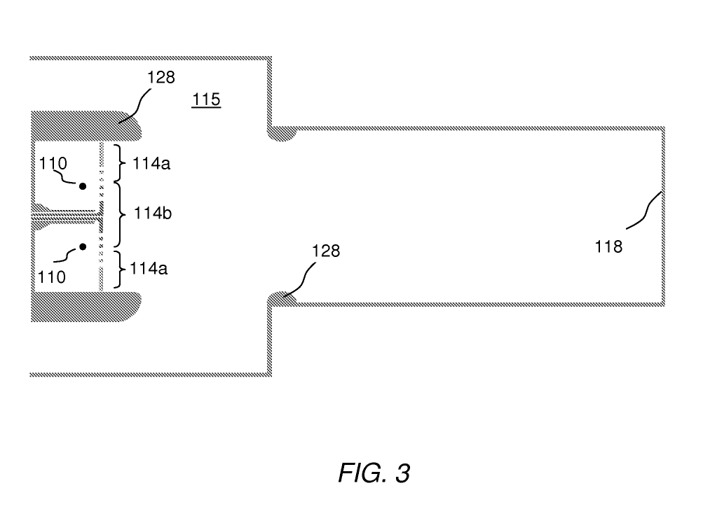

FIG. 3 illustrates an electron beam sterilizing device 102 according to a first embodiment of the present invention. The device has a construction which is similar to the device of FIG. 1, the main components being a filament 110, a grid 114 and an acceleration space leading to an output window 118. Also shown in FIG. 3 is a “beam shaper” 128, which may form part of the cathode housing. By affecting the electric field between the filament and the window with the beam shaper 128 the electron beam may be collimated properly (or focussed/defocused). The function of the beam shaper 128 is well known in prior art and several different variants are possible. In short, the purpose of the beam shaper is to shape the field accelerating the electrons, or in another way guide the electrons in their path. The beam shaper may comprise several components arranged prior to, and along the path of the electrons, which is why the same reference number has been given to several components. Generally, the cathode housing and its field shaping elements serves two purposes: Firstly, the shape and in particular the radii are designed such that the field strength is not excessive and secondly the shape and geometry of the raised elements 128 are designed such that the beam profile is optimal.

The main difference between the device of FIG. 3 and a prior art device is that the grid 114 comprises at least two operational portions. In the illustrated embodiment there is a radially inner grid 114b (inner grid in the following) and a radially outer grid 114a (outer grid in the following). The grids 114a, 114b are individually controllable by means of a voltage. This means that a variable voltage may be applied to either one, or both, of the grids 114a, 114b, in order to achieve a preferred beam configuration, e.g. a preferred beam profile.

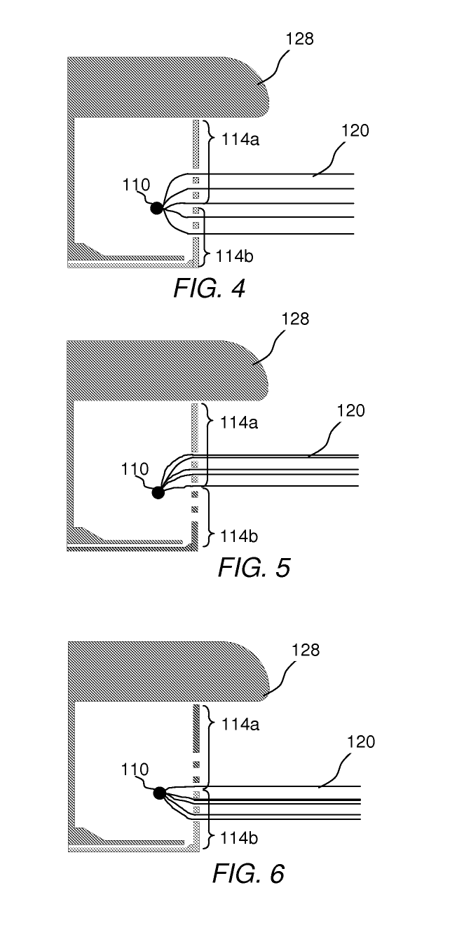

By controlling the inner 114b and outer grid 114a it is, in the illustrated embodiment, possible to create a small radius beam shape, by preventing electrons from passing through the outer grid 114a (see FIG. 6), an annular beam shape (doughnut-shaped profile), by preventing electrons from passing through the inner grid 114b (see FIG. 5), or a cylindrical beam shape (essentially homogenous), by allowing passage through both grids (see FIG. 4). The beam paths of the electrons are illustrated by the solid lines 120. It should be noted that the voltage applied to the grid 114a, 114b may be either positive or negative. Further, it should be noted that the voltage applied to the grid 114a, 114b is not very high, in the order of +/−100V. In the illustrated embodiment a voltage of −30-−40 V is used to efficiently block passage of electrons. This means that switching between different beam shape modes can be performed rapidly, basically as fast as the voltage can be switched, which makes the device very versatile.

It should be noted that if a specific degree of sterilization is to be achieved, it may be required to alter the filament power in order to achieve a satisfactory beam current (or anode current) as the electron beam device transfers between states. One obvious reason for this is that the area of the emitted beam profile may vary between different beam shapes, e.g. the small radius beam shape having a smaller cross sectional area than the annular beam shape. A practical example for one electron beam device is an anode current of 0.3 mA for the radially inner beam, and an anode current of 4 mA for the radially outer beam.

The grid 114 is made of any suitable electrically conductive and machinable material, generally a metal. In the illustrated embodiment stainless steel is used. The shape of the grid 114 is adapted to the desired shape of the resulting beam, and in general the grid is a metal plate equipped with holes or a wire mesh through which the electrons may pass. The solid portion of the grid 114 has the purpose of generating an electrical field with suitable properties and also has the purpose of adjusting the current from the filaments 110 by controlling the electric field strength at their surface. The holes may be circular, oblong, slit shaped, hexagonal (so as to give the grid a honeycomb shape) etc. Holes that are too large will result in that the electrons fan out, and consequently miss the exit window or deteriorate the distribution. If the holes are too small the high voltage field will not be able to “reach in” through the holes to collect the electrons in the desired fashion.

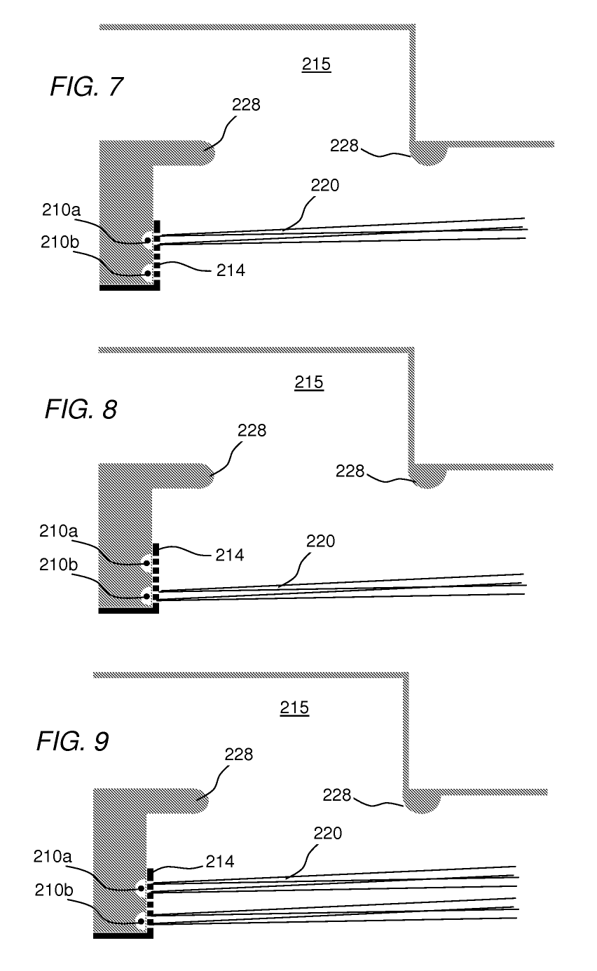

FIGS. 7-9 illustrate an alternative embodiment of the device, in which the filament 210 comprises at least two, individually controllable portions, a radially inner filament 210b and a radially outer filament 210a. The figures are partial sections including the filaments 210a, b, the grid 214 and a first region of the beam path. This embodiment allows for control of the beam shape and the beam current by control of the filaments 210a, 210b, similar to what was performed with the two grids 114a, 114b of the previous embodiment. FIG. 7 illustrates how the outer filament 210a is activated for an annular beam shape, FIG. 8 how the inner filament 210b is activated for the small radius beam shape and FIG. 9 how both filaments 210a, 210b are activated for the full, cylindrical, beam shape. The beam path of the electrons are illustrated by solid lines 220

In yet another embodiment the two previous embodiments may be combined to comprise two or more grids and two or more filaments, to achieve even better controllability. As such, a device designed in accordance with an embodiment of the invention may be space efficient, such that a high sterilization capacity may be contained in a limited space. Also, the filaments may be kept a constant optimal temperature, with optimal emission, between cycles.

It should be pointed out yet again that the invention is not limited to two filaments and/or grids. The number of individually operational filaments and/or grid may be varied within the physical constraints of the device in order to achieve the adequate performance of the resulting electron beam. One particular example is that a gradual shift from outer grids/filaments to inner grids/filaments could result in a more homogenous radiation of a sloping inner wall of a package, such as in a shoulder portion. The larger the sloping wall, the higher number of grids/filaments.

In use these embodiments will be used for the same purpose and in basically the same way. The possibility of varying the beam shape rapidly makes it possible to select a suitable beam shape for various parts of the package. As the device is translated into, or out of, the package the beam shape is adjusted to sterilize the particular part of the package that the device passes. For instance, when the device passes the body portion an annular beam shape may be used, by activation of the outer grid and/or outer filament. As the device approaches the shoulder portion the beam shape is switched to a homogenous profile by activating both grids and/or filaments. For sterilization of the neck and opening device the inner grid and/or filament is used. In this way an adequate sterilization can be achieved in all locations, without overexposure.

The transition between different beam profiles can be performed very fast, such that the sterilization device can operate without affecting the flow of a production line.

It is also possible to use alternative designs for the grids and filaments, deviating from the circular symmetry illustrated in the embodiments. The designs may suitably be varied to conform to the desired beam shape, and as such vary with the shape of the package to be sterilized.

Though the technical function of an electron beam sterilization device in general is considered to be known, the function of a device in accordance with the first embodiment will be described in some more detail in the following. The example given refers to the first embodiment.

Prior to sterilization the high-voltage field is applied. Negative voltage of about −40 V is applied to the outer and the inner grid, so as to prevent free electrons from passing through the grid. A current is fed through the filament, so as to heat it to approximately 2000° C., where the production of free electrons is sufficient. The device is inserted into a package to be sterilized. An alternative is to keep the device stationary, and thread the package over the device. Another alternative is to translate both the device and the package.

As the device is inserted into the package the potential of the outer grid is set to a higher value (which still may be, and generally is, 0 V or below), thus allowing an annular beam of electrons to be emitted from the output window so as to sterilize the inner walls of the body of the package. As the device approaches the shoulder portion of the package the potential of the inner grid is set to a higher value (which, as stated earlier, may still be negative) and the potential of the outer grid is reset to the lower −40 V, thus producing a small radius beam for sterilization of the cap portion. It should be noted that there may be an overlap so that both grids are at the higher potential during some period of time, if necessary in order to sterilize the tapered shoulder portion of the bottle. Both grids may be at the higher potential during insertion of the device, producing a full cylindrical beam instead of an annular one. As the device is retracted the above process is reversed. In an alternative sterilization process the device is only active during either insertion or retraction. It should be noted that the values given are highly dependent on the design of the electron beam device, and as such only constitute examples of possible values and not constraints, limiting the foreseeable values. In one design of the electron beam device the corresponding values for the lower and the higher potential are −150 V and −80 V, respectively.

In use the inventive device will be arranged in an irradiation chamber, i.e. a housing protecting the surrounding environment from radiation. Packages to be sterilized are brought into the irradiation chamber in such a way that leakage of irradiation is prevented in accordance to radiation design practice. This can be achieved by means of a lock gate, the interior design of the irradiation chamber and the function therein, or by only permitting entry of packages when devices inside the irradiation chamber are not emitting electrons.

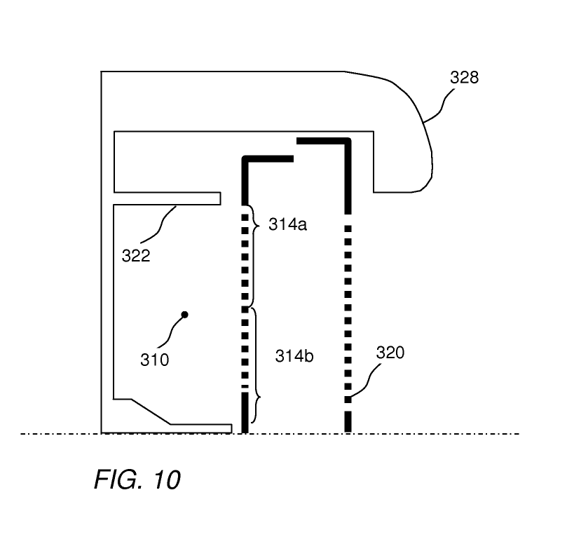

A third embodiment of the inventive device is described referring to FIG. 10. In this embodiment the switchable grid 314a, 314b is arranged between the filament 310 and an acceleration grid 320, which may be a grid with a constant grid voltage or variable grid voltage. The switchable grid comprises an outer extraction grid 314a and an inner extraction grid 314b. By applying a positive voltage of, e.g., 30-100 V (in this embodiment) to either one, or both, of the grids with respect to the filament, electrons emitted from the filament 310 will be attracted to that portion of the switchable grid, while a portion set at common will not attract any electrons. The purpose of the extraction grids is to distribute the emitted electrons in a specific spatial region. Once the electrons have passed the extraction grid they will be subjected to the electrical field from the acceleration grid 320, which results in them being accelerated at a right angle towards the acceleration grid 320. One or more field shaping elements, exemplified by element 322, may be arranged to affect the distribution of equipotential lines. The function of the inventive device according to this third embodiment is believed to be self-explanatory given the description of the previous embodiments. By varying the electrical potential of the filament grid it is possible to control the emission of electrons. It should be noted that the filament grid for this purpose may consist of more than two individually controlled sections. The shape of the switchable grid may also differ from what has been described. For example, the switchable grid may have a dome shape, essentially forming a semicircle around the filament.

In a forth embodiment, not shown, the filament grid is arranged on the far side of the filament, such that the switchable grid pushes rather than pulls the electrons towards the acceleration grid. Comparing with FIG. 10 this would correspond to a situation where the switchable grid 314a, b is arranged to the left of the filament 310. One advantage of this construction is that the transparency of the switchable grid will be infinite, since the electrons will not pass the actual grid, just be affected by its electrical field.

In yet another embodiment, not shown, only one grid is being used. The grid has two concentric sections covering one filament each, and e.g. the outer section has a lower reach through than the inner section. Consequently, at no grid voltage both beams would be on (broad beam). With increasing negative grid voltage the outer beam would be blocked first, the inner still being active (narrow beam). Later also the inner beam would be blocked (beam off). With such an arrangement the switching and current control functions could be done with only one grid power supply.

The type of package is arbitrary, but the device is particularly suited for sterilization of packages with a product contact surface (inner surface) comprising polymer. A RTF package generally comprises a body formed by a paper laminate sleeve provided with a plastic top. Yet, the device may also be used for sterilization of other products, such as medical equipment. The features of the inventive sterilization device make it very adaptable, such that tailor-made solution for packages of various shapes is simplified, so that each area of the package may be subject to an adequate radiation dose.

1. An electron beam sterilizing device, comprising;
an electron-generating filament,
a grid connected to a voltage source
a beam-shaper
an output window,
a high voltage source, capable of creating a high-voltage potential between the electron-generating filament and the output window, for acceleration of electrons,
wherein the electron-generating filament and/or the grid electrode comprises at least two individually operational portions for variation of the current and/or profile of an output electron beam.

2. The sterilizing device of claim 1, wherein the filament and/or the grid comprises two operational portions; a radially inner portion and a radially outer portion.

3. The sterilizing device of claim 1, wherein the peripheral shape of the filament and/or the grid is essentially circular; racetrack shaped; quadratic or rectangular, with or without rounded corners.

4. The sterilization device of claim 1, wherein the grid comprising two operational portions is arranged between the filament and an additional acceleration grid.

5. The sterilization device of claim 1, wherein the filament is arranged between the grid comprising two operational portions and the output window.

6. The sterilization device of claim 5, further comprising an acceleration grid between the filament and the output window.

7. The sterilization device of claim 1, wherein the device is adapted to sterilize a package.

8. The sterilization device of claim 5, wherein the package has a product contact surface comprising a polymer.

9. An electron beam sterilizing device comprising:
a housing in which is located a vacuum chamber, the housing comprising an outlet window;
a filament positioned in the vacuum chamber of the housing;
a power supply connected to the filament to drive current through the filament and generate electrons;
a grid connected to a voltage source;
a voltage supply configured to create a voltage potential between the filament and the exit window to accelerate the electrons generated by the filament, with the accelerated electrons passing out of the housing through the outlet window as an outlet electron beam to effect sterilization;
at least one of the filament and the grid comprising at least two individually operable portions to vary current and/or profile of the output electron beam; and
control means operatively connected to the at least two individuality operable portions to vary the current and/or profile of the output electron beam.

10. The sterilizing device of claim 9, wherein the at least two individually operable portions comprises a radially inner portion and a radially outer portion.

11. The sterilization device of claim 9, wherein the grid comprises two individually operable grid portions arranged between the filament and an additional acceleration grid.

12. The sterilization device of claim 9, wherein the filament is arranged between the grid, comprising two individually operable portions, and the output window.

13. The sterilization device of claim 12, further comprising an acceleration grid between the filament and the output window.

14. The sterilization device of claim 9, wherein the at least two individually operable portions comprises two individually operable grid portions each operatively connected to the control means which individually controls the voltage supplied to the two grid portions to supply a different voltage to the two grid portions.

15. The sterilization device of claim 9, wherein the at least two individually operable portions comprises two individually operable filament portions each operatively connected to the control means which individually controls the power supplied to the two filament portions to supply a different power to the two filament portions.

16. A method of controlling sterilization of a packaging laminate through use of an electron beam sterilizing device, the electron beam sterilizing device comprising a housing in which is positioned a filament connected to a power supply, the housing possessing an output window, the method comprising:
positioning the packaging laminate at the output window while drive current, sufficient to cause the filament to generate and emit electrons, is delivered to the filament from the power supply;
accelerating the electrons generated by the filament so that an electron beam passes through the exit window and impinges on a surface of the packaging laminate to sterilize the surface of the packaging laminate; and
individually operating at least two individually operable portions of the filament and/or the grid to vary current and/or profile of the electron beam.
